SEQUENCE LISTING (14 sequences)

SEQ ID NO: 1 - protein, 4 aa, Artificial Sequence
  Synthetic peptide
GHRP

SEQ ID NO: 2 - protein, 4 aa, Artificial Sequence
  Synthetic peptide
KHRR

SEQ ID NO: 3 - protein, 4 aa, Artificial Sequence
  Synthetic peptide
AAAA

SEQ ID NO: 4 - protein, 7 aa, Artificial Sequence
  Synthetic peptide
GHRPSYQ

SEQ ID NO: 5 - protein, 7 aa, Artificial Sequence
  Synthetic peptide
GHRPSYQ

SEQ ID NO: 6 - protein, 6 aa, Artificial Sequence
  Synthetic peptide
GDRPSY

SEQ ID NO: 7 - protein, 4 aa, Artificial Sequence
  Synthetic peptide
PSYQ

SEQ ID NO: 8 - DNA, 35 bp, Artificial Sequence
  Synthetic primer
cggcctatgacctctagctcctcttctgaatcggg

SEQ ID NO: 9 - DNA, 31 bp, Artificial Sequence
  Synthetic primer
ggtcataggccgtcatatcaaggaaacagtg

SEQ ID NO: 10 - DNA, 18 bp, Artificial Sequence
  Synthetic primer
gatctgccaccatggatg

SEQ ID NO: 11 - DNA, 25 bp, Artificial Sequence
  Synthetic primer
tggtctagatcacggtcgcatgttg

SEQ ID NO: 12 - DNA, 29 bp, Artificial Sequence
  Synthetic primer
agcgaagattgcagcctgccaggggtggg

SEQ ID NO: 13 - DNA, 41 bp, Artificial Sequence
  Synthetic primer
caatcttcgctgctgcagctgcctcgccaggagaaaggttc

SEQ ID NO: 14 - DNA, 24 bp, Artificial Sequence
  Synthetic primer
agatctgccaccatggatgcaatg

(19) United States
(12) Patent Application Publication
(10) Pub. No.: US 2012/0058537 A1
(43) Pub. Date: Mar. 8, 2012

(54) CHIMERIC TRUNCATED AND MUTANT VARIANT OF TISSUE PLASMINOGEN ACTIVATOR (T-PA) RESISTANT TO PLASMINOGEN ACTIVATOR INHIBITOR-1

(75) Inventors: Fereidoun Mahboudi, TEHRAN (IR); Fatemeh Davami, TEHRAN (IR); Soroush Sardari, Tehran (IR)

(73) Assignees: Fereidoun Mahboudi, Tehran (IR); PASTEUR INSTITUTE OF IRAN (IPI), No. 69, TEHRAN (IR); Soroush Sardari, Tehran (IR); Fatemeh Davami, Tehran (IR)

(21) Appl. No.: 13/191,933

(22) Filed: Jul. 27, 2011

(51) Int. Cl. C12N 9/50

(52) U.S. Cl. 435/219

(57) ABSTRACT

The various embodiments herein provide a chimeric truncated and mutant variant of a tissue plasminogen activator (t-pa) and a method for preparing the same. According to an embodiment herein, the mutant variant comprises a signal sequence domain, followed by a chimeric tetrapeptide, followed by a tripeptide, followed by a kringle 2 domain, followed by a serine protease domain and a substituted amino acids at position 128-131. The substituted amino acids are AAAA (SEQ ID NO: 3) amino acids. The chimeric tetrapeptide is Gly-His-Arg-Pro (SEQ ID NO: 1). The chimeric tetrapeptide is at a position of 36 to 39 amino acid of the mutant variant. The tripeptide is Ser-Tyr-Glu. According to an embodiment herein, a chimeric truncated and mutant variant of a tissue plasminogen activator comprises a native t-pa deleted with Finger domain, a Growth Factor domain and a Kringle 1 domain, a chimeric tetrapeptide and a substituted amino acids at a position of 128-131.

### SPONSORSHIP STATEMENT

The present invention is sponsored by Pasteur Institute of Iran (IPI).

### BACKGROUND

1. Technical Field

The embodiments herein generally relate to the field of thrombolytic drugs and particularly to tissue plasminogen activators (t-PA). The embodiments herein more particularly relate to a novel variant of tissue plasminogen activator (t-PA) with improved pharmacodynamic properties compared to native tissue plasminogen activator.

2. Description of the Related Art

Coronary heart diseases including myocardial infarction have a significant part (52%) on percentage of death caused by cardiovascular diseases. Accordingly, the treatment of ischemic stroke is one of the most challenging areas in medicine today. Myocardial infarction (MI) is an irreversible necrosis or death of heart muscles due to prolonged ischemia. According to the third monitoring report of the World Health Organization, cardiovascular diseases cause 12 million deaths throughout the world each year. Thrombolytic drugs play a crucial role in the management of patients with acute myocardial infarction, pulmonary embolism, deep vein thrombosis, arterial thrombosis, acute thrombosis of retinal vessel, extensive coronary emboli, and peripheral vascular thrombo-embolism. Recognition of the importance of fibrinolytic system in thrombus resolution has resulted in the development of various fibrinolytic agents and plasminogen activators (PAs) with different pharmacokinetic and pharmacodynamic properties. Three different generations of PAs have been introduced to the market. The first generation agents are Streptokinase and Urokinase. The second generation agents are Alteplase and Acylated plasminogen streptokinase activator complex (APSAC). The third generation agents are Vampire bat plasminogen activator (BatPA), Reteplase, Tenecteplase, Lanatoplase, and Staphylokinase.

Plasminogen activators are of great clinical significance as thrombolytic agents for management of stroke and myocardial infarction. Tissue-type plasminogen activator (t-PA) is generally preferred for its more efficacy and safety compared to urokinase and streptokinase. Tissue-type plasminogen activator (t-PA) is a glycoprotein consisting of 527 amino acid residues (72 KDa) with seventeen disulfide bonds and approximate 7% carbohydrate in total molecular weight. Enhanced activity in the presence of fibrin, i.e. fibrin-specific plasminogen activation is the major advantage of t-PA over other thrombolytic agents. Tissue-type plasminogen activator (t-PA), mainly released by endothelial cells, cleaves the zymogen plasminogen into active plasmin. Plasmin degrades fibrin, as the major component of clots, and promotes blood reperfusion. Type-1 plasminogen-activator inhibitor (PAI-1) and a2-antiplasmin (a2-AP) can inhibit this cascade by blocking the proteolytic activity of t-PA and plasmin, respectively. PAI-1 belongs to serpin family which plays its role as an ideal pseudo-substrate for target serine proteases. The first source of PAI-1 is synthesized by endothelial cells and/or by hepatocytes. The second pool of PAI-1 is contained within the α-granules of platelets. The interaction between t-PA and PAI-1 bound to fibrin is composed of three sequential steps: (a) interaction of the catalytic site of t-PA with the reactive center of PAI-1, bound to fibrin, (b) conformational change in the complex that leads to loss of its affinity for fibrin, and (c) dissociation from the fibrin matrix and rebinding to fibrin subsequently; that would greatly impede t-PA activity. Development of various forms of t-PA (e.g. Alteplase®, Rete-plase® and Tenecteplase®) has exploited the activity of t-PA. Since the recognition that residues 296-304 are critical for the interaction of t-PA with PAI-1, several variants of t-PA with mutations or deletions in this domain have been investigated.

Tissue-type plasminogen activator (t-PA) is the dominant PA involved in fibrinolysis. T-PA is a glycoprotein with 67 kDa, 527 amino acids, which promotes conversion of plasminogen to plasmin in the presence of fibrin. The protein molecule is divided into five structural domains: finger domain (F) followed by a growth factor domain (EGF) near the N-terminal region and the two kringle 1 (K1) and kringle 2 (K2) domains. Next to kringle 2 domain is the serine protease domain with the catalytic site at the C terminus. Both finger and kringle 2 bind to the fibrin and accelerate t-PA activation on plasminogen. However, full length t-PA has some major disadvantages among which the rapid clearance from plasma due to the recognition of structural elements on first three N-terminal domains by certain hepatic receptors is the most important. Human fibrinogen can be converted to fibrin through thrombin-catalyzed release of small peptides from the amino-terminal segments of the K and L chains that are named fibrinopeptides A and B, respectively. The tetrapeptide GHRP can interact with a complementary site on the L lobe of fibrin monomers and prevent polymerization. Furthermore, it has been reported that histidine-16 of the BL chain plays an important role in the association of fibrin.

Furthermore, prokaryotic production and refolding process of full length form is challenging. These two substantial reasons have lead to synthesis of the smallest active molecule such as Reteplase and Lanatoplase that are commercially available. Reteplase is a deletion mutant of t-PA with prolonged half-life, in which the F, EGF, and K1 region of wild type t-PA molecule have been deleted. Finger domain is the responsible domain for fibrin affinity. Therefore, Reteplase has weaker affinity for fibrin and causes more fibrinogen depletion than full length forms.

Resistance to PAI-1 is another factor which confers clinical benefits in thrombolytic therapy. The only US FDA approved PAI-1 resistant drug is Tenecteplase®. Deletion variants of t-PA have the advantage of fewer disulfide bonds in addition to higher plasma half lives.

Development of various forms of t-PA (e.g. Alteplase®, Rete-plase® and Tenecteplase®) has exploited the activity of t-PA. Since the recognition that residues 296-304 are critical for the interaction of t-PA with PAI-1, several variants of t-PA with mutations or deletions in this domain have been investigated. Tenecteplase® is the only FDA approved PAI-1 resistant thrombolytic agent. Tenecteplase® consists of two point mutations at positions 103, 117 that causes prolonged plasma half life. Furthermore, the four amino acids at position 296-299 have been replaced by four alanines which make resistance towards inhibition by PAI-1. Reteplase® is a single-chain non-glycosylated deletion variant of t-PA consisting of only the second kringle and the protease domains. Since finger domain is the responsible domain for fibrin affinity, Reteplase® is characterized by reduced fibrin selectivity and causes more fibrinogen depletion than the full length forms. In the absence of fibrin, Reteplase and Alteplase do not differ with respect to their activity as plasminogen activators, nor do they differ in terms of their inhibition by the PAI-1.

Hence there is a need to develop a variant of tissue plasminogen activator (t-pa) that has more fibrin activity and is resistant to plasminogen activator inhibitor-1.

The above mentioned shortcomings, disadvantages and problems are addressed herein and which will be understood by reading and studying the following specification.

### OBJECTIVES OF THE EMBODIMENTS

The primary object of the embodiments herein is to provide a mutant variant of tissue plasminogen activator (t-pa) that is resistant to plasminogen activator inhibitor-1.

Another object of the embodiments herein is to provide a variant of tissue plasminogen activator (t-pa) with increased fibrin activity.

Yet another object of the embodiments herein is to provide a variant of tissue plasminogen activator (t-pa) that has a rapid clearance from the plasma.

Yet another object of the embodiments herein is to provide a variant of tissue plasminogen activator (t-pa) that does not cause much depletion of fibrinogen compared to the full length forms of t-pa.

Yet another object of the embodiments herein is to provide a variant of tissue plasminogen activator (t-pa) that forms a promising approach with more desirable pharmacodynamic properties compared to existing commercial forms.

Yet another object of the embodiments herein is to provide a variant of tissue plasminogen activator (t-pa) as the first PAI-1 resistant truncated t-PA, to provide high PAI-1 levels to make the thrombolytic system prone to re-occlusion.

These and other objects and advantages of the embodiments herein will become readily apparent from the following detailed description taken in conjunction with the accompanying drawings.

### SUMMARY

The various embodiments herein provide a novel truncated mutant variant of tissue plasminogen activator or t-pa wherein the first three domains of native t-PA are deleted, a chimeric tertapeptide Gly-His-Arg-Pro (GHRP) is added and the KHRR 128-131 amino acids are substituted with AAAA amino acids. The chimeric tertapeptide Gly-His-Arg-Pro (GHRP) is added to the upstream of kringle 1 and serine protease domain (K2S domain) of the truncated mutant variant of t-pa. The mutant variant in the embodiments herein has an increased half life and a high fibrin affinity. The increased half life is due to the deletion of the first three domains of the full length t-pa. The domains deleted are Finger domain (F), growth factor domain (EGF) and kringle 1 domain (K1) of the native full length t-pa. The addition of the chimeric tertapeptide Gly-His-Arg-Pro (GHRP) is responsible for the high fibrin affinity. The chimeric tertapeptide Gly-His-Arg-Pro (GHRP) added upstream of K2S domain compensates for the diminished fibrin affinity due to F domain deletion.

The specific activity of the novel truncated mutant variant, in the embodiments herein, is 570 IU/μg and an 85% of residual activity after inhibition by rPAI-1. The new variant in the embodiments herein, as the first PAI-1 resistant truncated t-PA, offers more advantages in clinical conditions in which high PAI-1 levels makes the thrombolytic system prone to re-occlusion.

According to an embodiment herein, a chimeric truncated and mutant variant of a tissue plasminogen activator (t-pa) comprises a native t-pa deleted with a first three domains, a chimeric tetrapeptide and a substituted amino acids at a position of 128-131. The mutant variant is resistant to plasminogen activator inhibitor-1. The deleted first three domains are Finger domain (F), a Growth Factor domain (EGF) and a Kringle 1 domain (K1). The chimeric tetrapeptide is Gly-His-Arg-Pro (GHRP), and the chimeric tetrapeptide is at a position of 36 to 39 amino acid of the mutant variant. The chimeric tetrapeptide (GHRP) is situated on the N-terminus. The substituted amino acids are AAAA amino acids. The mutant variant has 394 amino acids. The mutant variant has a fibrin affinity of 86%. The mutant variant has a specific activity of is 570 IU/μg. The mutant variant has a residual activity of 85% after inhibition by plasminogen activator inhibitor-1.

According to an embodiment herein, a method for preparing a chimeric truncated and mutant variant of t-pa comprises deleting first three domains of a native t-pa, adding a chimeric tetrapeptide and substituting amino acids KHRR with amino acids AAAA at position 128-131 amino acid of the native t-pa. The first three deleted domains of the native t-pa are a Finger domain (F), a Growth Factor domain (EGF) and a Kringle 1 domain (K1). The chimeric tetrapeptide is Gly-His-Arg-Pro (GHRP) and is added at a position of 36 to 39 amino acid of the mutant variant. The mutant variant is produced by using a Splicing by Overlap Extension PCR (SOEing-PCR) method. The chimeric tetrapeptide (GHRP) is added upstream to a kringle 2 domain and a serine protease domain (i.e. K2S domain) of the native t-pa. The chimeric tetrapeptide compensates for diminished fibrin affinity due to F domain deletion from a native t-pa. The mutant variant has 394 amino acids. The chimeric tetrapeptide (GHRP) has a high fibrin affinity. The mutant variant has a specific activity of 570 IU/μg. The mutant variant has a residual activity of 85% after inhibition by plasminogen activator inhibitor-1.

According to an embodiment herein, a chimeric truncated and mutant variant of a tissue plasminogen activator (t-pa) comprises a native tissue plasminogen activator (t-pa) having a signal sequence domain followed by a chimeric tetrapeptide and then followed by a tripeptide, and afterwards followed by a kringle 2 domain and finally followed by a serine protease domain and a substituted amino acids at position 128-131. The substituted amino acids are AAAA amino acids. The mutant variant is resistant to plasminogen activator inhibitor-1. The chimeric tetrapeptide is Gly-His-Arg-Pro (GHRP) at a position of 36 to 39 amino acid of the mutant variant. The tripeptide is Ser-Tyr-Glu (SYQ). An arrangement of the tripeptide, the kringle 2 domain and the serine protease domain (SYQ-K2S) is responsible for the serine protease activity.

According to one embodiment herein, the chimeric truncated and mutant variant of a tissue plasminogen activator (t-pa) comprises a signal sequence domain, followed by a chimeric tetrapeptide, followed by a tripeptide, followed by a kringle 2 domain, followed by a serine protease domain and a substituted amino acids at position 128-131 with AAAA amino acids. The chimeric tetrapeptide is Gly-His-Arg-Pro (GHRP). The chimeric tetrapeptide is at a position of 36 to 39 amino acid of the mutant variant. The tripeptide is Ser-Tyr-Glu (SYQ). The mutant variant in the embodiments herein is resistant to tissue plasminogen inhibitor-1. The mutant variant has a total of 394 amino acids. The mutant variant is produced by using Splicing by Overlap Extension PCR (SOEing-PCR) methodology. The first three domains of a native t-pa are deleted and amino acids KHRR at 128-131 position of the native t-pa are substituted with AAAA amino acids to obtain the mutant variant. The deleted domains are a Finger domain (F), a growth factor domain (EGF) and a Kringle 1 domain. The chimeric tetrapeptide (GHRP) is added upstream to the kringle 2 domain and the serine protease domain (i.e. K2S domain). The chimeric tetrapeptide (GHRP) is situated on the N-terminus. The chimeric tetrapeptide (GHRP) has a high fibrin affinity. The specific activity of the mutant variant is 570 IU/μg. The arrangement of the tripeptide, the kringle 2 and the serine protease domains (SYQ-K2S) is responsible for the serine protease activity. The chimeric tetrapeptide compensates for diminished fibrin affinity due to F domain deletion from a native t-pa. The truncated mutant form shows a residual activity of 85% after inhibition by Plasminogen activator inhibitor-1. The mutant variant in the embodiments herein, has an increased half-life.

The novel truncated form of t-PA, in the embodiments herein, is expressed in Chinese hamster ovarian (CHO) cells.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a block diagram indicating the general mechanism of formation of the novel truncated mutant by Splicing by overlap extension PCR method, according to one embodiment herein.

FIG. 2A shows a cartoon ribbon display for two different views of GHRP-SYQ-K2S modeled with MODELLER 9V1 indicating a left back view of a ribbon model of GHRP-SYQ-K2S modeled with MODELLER 9V1 automodel package.

FIG. 2B shows a cartoon ribbon display for two different views of GHRP-SYQ-K2S modeled with MODELLER 9V1 indicating a left bottom view for a ribbon model of GHRP-SYQ-K2S modeled with MODELLER 9V1 automodel package.

FIG. 3 shows a result of a Gel-electrophoresis analysis of PCR-products of Chimeric truncated t-PA.

FIG. 4 shows a result of a Gel-electrophoresis analysis of PCR products performed on stable transfected CHO cells and control cell line.

FIG. 5A shows a result of a SDS-PAGE analysis of supernatant of transfected CHO cells.

FIG. 5B shows a result of a Western Blot analysis of supernatant of transfected CHO cells.

FIG. 6 shows plasminogen activators and proteases in polyacrylamide gel containing gelatin after an electrophoretic activity analysis of the transfected t-pa and gels.

FIG. 7 shows a graphical representation of the fibrin affinity assay of full length and chimeric truncated t-pa indicating binding to fibrin clots at various concentrations of fibrinogen.

FIG. 8A shows a Cartoon Ribbon display in VMD 1.8.7 program for modified t-PA modeled with MODELLER 9V1 indicating a Left top back view of a ribbon model of modified t-PA modeled with MODELLER 9V1-auto model package.

FIG. 8B shows a result of the Ramachandran plot analysis for the modified t-PA modeled with MODELLER 9V1.

FIG. 9 shows a result of a Gel-electrophoresis analysis of PCR-products of Chimeric-Mutant t-PA on 1% agarose gel.

FIG. 10 shows a result of a Gel-electrophoresis analysis of PCR-products performed on stable transfected CHO cells and control cell line.

FIG. 11 shows a chart indicating a relationship between a purification of chimeric truncated mutant t-PA on affinity chromatography HiTrap™ Benzamidine FF column.

FIG. 12A shows a result of a SDS-PAGE analysis of purified truncated-mutant t-PA.

FIG. 12B shows a result of a Western Blot analysis of purified truncated-mutant t-PA.

FIG. 13 shows a presence of plasminogen activators in 11% polyacrylamide gel containing gelatin and plasminogen, after an electrophoresis activity of gel.

FIG. 14 shows an expression of truncated-mutant t-PA in CHO DG44 supernatant versus viability after an Amidolytic assay of Chromolize™ performed to determine the expression kinetic of modified t-PA.

FIG. 15 shows a chart indicating a relationship between a residual activity of full length and PAI-1 resistant Truncated Mutant t-PA after inhibition by rPAI-1.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The embodiments herein provide a novel truncated mutant of tissue plasminogen activator (t-pa) developed by deletion of the first three domains in t-PA in addition to substitution of KHRR 128-131 amino acids with AAAA in truncated t-PA.

Tissue plasminogen activator (t-PA) is one of the fibrin-specific serine proteases that play a crucial role in the fibrinolytic system. The rapid clearance of the drug from the circulation, caused by its active uptake in the liver, has lead to complicated clinical applications. Different forms of plasminogen activators have been developed to treat thrombotic disease. Deletion of the first three domains of t-PA by gene manipulation techniques has shown a significant increase in its plasma half life. In order to compensate the disadvantage of higher bleeding risk, a novel chimeric truncated form of t-PA with 394 amino acids and more fibrin affinity compared to the truncated form is designed and expressed in Chinese Hamster Ovarian (CHO) cells.

According to the embodiments herein, the recombinant chimeric plasminogen activator consists of kringle 2 and serine protease (K2S) domains of t-PA, namely GHRP-SYQ-K2S. The level of expression is 752 IU/ml with 566,917 IU/mg specific activity, based on amidolytic activity. The fibrin binding of this novel chimeric truncated t-PA is 86% of the full length t-PA at a fibrinogen concentration of 0.2 mg/ml.

The deletion of first three domains of t-PA eliminate clearance sites and increases half life, furthermore, a chimeric tetra-peptide Gly-His-Arg-Pro (GHRP) with high fibrin affinity, added to the upstream of K2S (Kringle 2 serine) domain compensates for reduction of fibrin affinity due to finger domain deletion. The PAI-1 resistant novel form of truncated t-PA in the embodiments herein, is designed based on In Silico modeling. The PAI-1 resistant novel form of truncated t-PA is successfully expressed in CHO DG44. Targeting mutations made, according to embodiments herein, increase the resistance of truncated t-PA to inhibition by PAI-1.

### Experimental Data

The novel truncated form t-pa is expressed in Chinese Hamster Ovarian (CHO).

### Materials and Methods

DG44 transfection kit and Zeocin selection marker were obtained from Gibco-Invitrogen cooperation (CA, USA). Plasminogen was received from Sigma (MO, USA); Chromolize t-PA Assay Kit was purchased from Biopool (Trinity Biotech plc. Ireland). Rabbit polyclonal antibody to t-PA was supplied by Abcam (MA, USA) and goat anti rabbit IgGHRP conjugated was purchased from Santa Cruz biotechnology (CA, USA). All reagents for SDS/polyacrylamide gel electrophoresis were from Bio-Rad, (CA, USA). All of the molecular modeling and structure visualizations were done on a dual processor windows-based platform, workstation using the Deepview/Swiss PDB viewer program version 3.7, 2001 (http://www.expasy.org/spdbv) and VMD 1.8.7 (http://www.ks.uiuc.edu), DPM (double prediction Method) algorithm. Secondary structure prediction was used to predict the secondary structure of our novel protein. Atomic coordinates of kringle 2 and serine protease domain of t-PA were retrieved from the Protein Data Bank (http://www.rcsb.org/pdb/cgi) [entries 1tpk and 1bda, respectively] and 1fzv and 2flh both were used as templates for the 7 amino acid chimeric part: GHRP-SYQ. The 1fzv and 2flh entries were chosen since they had GDRPSY and PSYQ in their sequence, respectively, and their pdb files did not cause any problem. To produce theoretical chimeric truncated t-PA using above templates a model was designed by Modeller 9v1, via automodel package. Energy minimization was done by GROMOS96 implementation of Swiss-Pdbviewer http://www.igc.ethz.ch/GROMOS/index). The Ramachandran plots analysis was performed by RAMPAGE online Ramachandran plot analysis software (http://mordred.bioc.cam.ac.uk/rapper/rampage). A dual processor windows based platform workstation was utilized to perform all the molecular modeling and structure visualizations via the Deep-view/Swiss PDB viewer program version 3.7, 2001 (www.expy.org/spdbv), VMD 1.8.7 (http://www.ks.uiuc.edu) and DPM (Double Prediction Method) algorithm for secondary structure prediction. Protein Data Bank (www.rcsb.org/pdb/cgi) was the source for retrieving all atomic coordinates of t-PA domains. The model was designed using auto model package of Modeller 9v1 software. Energy minimization was done by GROMOS96 implementation of Swiss-PDB-viewer (http://iqc.ethz.ch/gromos). RMSD values were calculated by superimposing modeled protein structure on different PDB templates for Kringle 2 and serine protease domains. The Ramachandran plots analysis was performed by RAMPAGE software (http://mordred.bioc.cam.ac.uk/˜rapper/rampage) to ensure model validity.

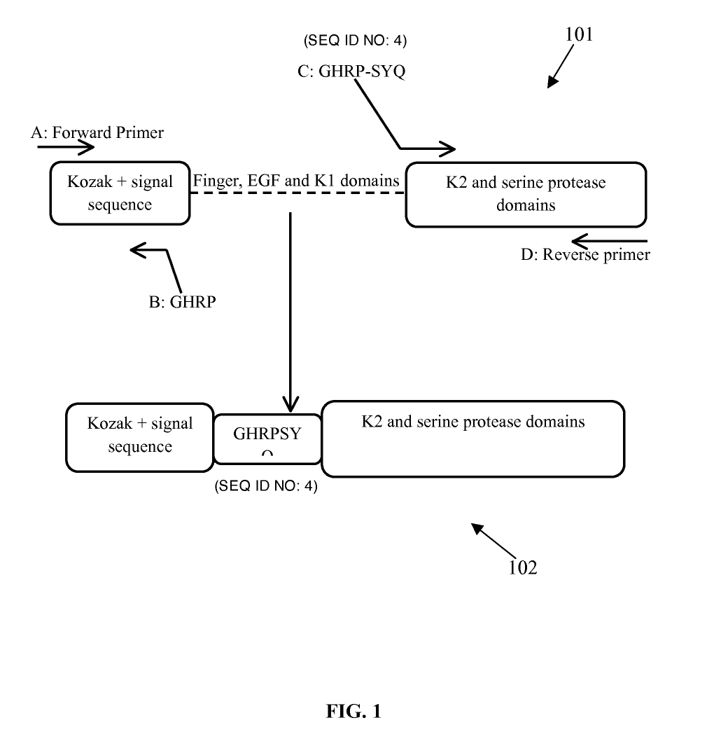

FIG. 1 shows a block diagram indicating the general mechanism of formation of the novel truncated mutant by Splicing by Overlap Extension (SOE)—Polymerase Chain Reaction (PCR) method, according to one embodiment herein. The desired gene cassette was constructed using Splicing by Overlap Extension PCR i.e. SOEing-PCR. Full-length human t-PA 101, GenBank accession number 101047, was amplified using the CHO 1-15 cell line i.e. ATCC-CRL 9606 genomic DNA and cloned into pTZ57R. The segments to be joined were fragments AB and CD. The fragment AB was joined from the signal sequence from upstream of t-PA full length gene 101 and the fragment CD was joined from the K2S end from the downstream of the gene. The fragments were amplified in separate PCRs i.e. “1” and “2”. The primers used to be in “SOEn” are B and C, wherein B is CGGCCTATGACCTCTAGCTCCTCTTCTGAATCGGG and C is GGTCATAGGCCGTCATATCAAGGAAACAGTG, according to the embodiments herein. The primers were made partially complementary to one another and nucleotides i.e. GHRP-SYQ to be inserted between signal sequence and K2S were included. The Forward and Reverse primers, with respect to FIG. 1, were A and D, wherein A is GATCTGCCACCATGGATG, and wherein D is TGGTCTAGATCACGGTCGCATGTTG. The PCR products of these first reactions overlap via their homologous sequences at the ends that contain the desired insertion GHRP-SYQ as well. The three domains were deleted and a 21 bp fragment was inserted simultaneously. The products of the first step were mixed in a SOE reaction (reaction 3), and subjected to repeated rounds of denaturation, reannealing, and primer extension by DNA polymerase. By the presence of the appropriate primers (“A” and “D”) in the reaction, the recombinant product 102 was formed.

According to the embodiments herein, the step of SOEing-PCR was done in the following conditions for AB and CD fragments: first reaction: 1 μL t-PA DNA (22.7 ng/μL), 0.2 mM DNTP, 1 μL primer A (0.4 μM), 2 μL primer B (0.4 μM), 21 μL Buffer, and 2 mM MgSO4; Second reaction: 1 μL t-PA DNA (26 ng/μL), 0.2 mM DNTP, 1 μL primer D (0.4 μM), 2 μL primer C (0.4 μM), 21 μL Buffer, and 2 mM MgSO4.

According to one embodiment herein, the SOEing PCR reaction was as follows: Fragment AB as the first template 1 μL (54 ng/μL) and fragment CD as second template 1 μL (58 ng/μL) were combined and the PCR reaction was run without the presence of primers for 3 cycles (in the first cycle, the reaction was carried out at 94° C. for 4 min, and in the second cycle, the reaction was carried out at 94° C. for 1 min and in the third cycle, the reaction was carried out at 72° C. 1 min), then the procedure was completed y treating the sample at 94° C. for 1 min, at 68° C. for 1 min and at 72° C. for 1 min. this process was repeated for 30 cycles, and a final 5 min heating at 72° C. was performed at the extension time in the presence of A and D primers.

Further, a full length human t-PA (GenBank accession number I01047) was amplified using the CHO 1-15 cell line (ATCC-CRL 9096) genomic DNA and cloned into pTZ57R. Amino acid substitution, KHRR to AAAA, at position 128-131 was performed using SOEing PCR via appropriate primer design in a three step reaction utilizing truncated gene cloned previously in a plasmid as DNA template for desired mutations. The primers used to be “SOEn” (B: AGCGAAGATTGCAGCCTGCCAGGGGTGGG and C: CAATCTTCGCTGCTGCAGCTGCCTCGCCAGGAGAAAGGTTC) were made partially complementary to one another and also include nucleotides to be mutated between the upstream and the downstream fragments. Forward and reverse primers were, as follows: A: AGATCTGCCACCATGGATGCAATG, and D: TGGTCTAGATCACGGTCGCATGTTG. The PCR products of these first reactions overlap via their homologous sequences at the ends which contain desired mutation (from KHRR to AAAA). The products of the first step were mixed in a SOEing reaction, and in the presence of the appropriate primers (‘A’ and ‘D’), the recombinant product was formed.

The first step PCR reactions was done in the following conditions for AB and CD fragments: First reaction: 1 μl t-PA DNA (35.7 ng/μl), 0.2 mM DNTP, 1 μl primer A (0.4 μM), 2 μl primer B (0.4 μM), 21 μl Buffer, 2 mM MgSO4, Second reaction: 1 μL t-PA DNA (37 ng/μl), 0.2 mM DNTP, 1 μl primer D (0.4 μM), 2 μl primer C (0.4 μM), 21 μl Buffer, 2 mM MgSO4. The SOEing PCR reaction was as follows: Fragment AB as the first template 1 μl (62 ng/μl) and fragment CD as second template 1 μl (58 ng/μl) were combined and the PCR reaction was run in the absence of primers for 3 cycles (at 94° C. for 4 min, at 94° C. for 1 min and at 72° C. 1 min). The procedure was completed by treating at 94° C. for 1 min, 68° C. for 1 min, 72° C. for 1 min for 30 cycles, and a final 5 min 72° C. extension time in the presence of A and D primers.

After performing SOEing PCRs, the SOEn 1200 bp gene was cleaned up by a QIAquick PCR Purification kit (Qiagen Germany), cloned in an intermediate vector pJET1.2/blunt Cloning Vector via CloneJET PCR Cloning Kit (Fermentas, Vilnius, Lithuania) based on the manufacturer's procedures. After confirming the proper sequence arrangement by bidirectional sequencing, two upstream and downstream BglII restriction sites of pJET were used due to accomplish cloning into CHO expression vector, EcoRV site in pTracer-SV40's. BglII sticky ends were converted to blunt ends using CloneJET PCR Cloning Kit DNA blunting enzyme. The recombinant pTracer-SV40-chimeric-truncated-t-PA plasmid was purified by EndoFree Plasmid Mega kit (Qiagen Germany) and the presence and right orientation of the gene was confirmed by SmaI restriction enzyme digestion.

The SOEn gene was cloned in EcoRV site in pTracer-SV40's (CHO expression vector). The recombinant pTracer-SV40-mutated t-PA plasmid was purified by EndoFree™ Plasmid Mega kit (Qiagen Germany) and the right orientation of the gene was confirmed by SmaI restriction enzyme digestion and sequencing analysis. DG44 Transfection Kit (GIBCO Invitrogen USA) was utilized to transfect DG44 CHO cells by LIPOFECTAMINE 2000 CD, based on manufacturer's 12 well plate's protocol. Transfected cells were examined under fluorescent microscope GFP filters with excitation wavelength of 400 nm and emissions wavelength of 510 nm for transient expression of pTracer SV40 GFP protein which is partially correlated with t-PA gene expression.

Zeocin sensitivity of CHO DG44 cells was determined based on pTracer-SV40 manual guide. The pTracer-SV40-chimeric-truncated t-PA was linearized from BglII restriction site. Linearized plasmid was then transected to the cells by DG44 transfection kit, and stable integrants were selected above the predetermined Zeocin sensitivity concentration (500 μg/mL).

Zeocin™ sensitivity of CHO DG44 cells was determined based on pTracer-SV40 manual guide. The pTracer-SV40-mutated t-PA was linearized from BglII restriction site. Linearized plasmid was then transfected to the cells by DG44 transfection kit through lipofection by Lipofectamine 2,000, and stable integrants were selected above the predetermined Zeocin™ sensitivity concentration (500 μg/ml)

Supernatants from stable transfected CHO culture medium were harvested and concentrated using Amicon filtering system (Millipore, USA). SDS-PAGE was carried out in a 12% resolving polyacrylamide gel according to the Laemmli method. Western blot analysis of culture media was performed according to Sambrook et al. Electroblotting was performed in a semidry blotting system. Proteins were transferred to a nitrocellulose membrane, and antigen-antibody complexes were visualized by DAB-HRP system. Primary polyclonal rabbit anti-t-PA antibody (Abcam. USA) were diluted 1/1000, the secondary antibody, peroxidase conjugated goat antirabbit antibody (Santa Cruz. USA) was used in a 1/1500 dilution.

An 11% resolving polyacrylamide gel was copolymerized with plasminogen and gelatin as previously described by Heussen and Dowdle. The stacking gel was prepared as 4% concentration without plasminogen and gelatin. Electrophoresis was performed at 40° C. at a constant current of 8 mA. The residual SDS in gel slab was removed after a gentle shaking at room temperature for 1 h in 2.5% Triton X-100. Then, the gel slab was incubated in 0.1 M glycine-NaOH, pH 8.3, for 5 h at 37° C. Finally, the gel slab was stained and destained by standard Coomassie brilliant blue (R-250) dying system. The location of the peptide harboring enzymatic activity was not stained by dye in contrast to blue-paint background.

Polyacrylamide gel (11%) was copolymerized with plasminogen and gelatin as previously described by Heussen et al. The stacking gel was prepared without plasminogen and gelatin. A constant current of 8 mA was performed at 4° C. for electrophoresis. The residual SDS was removed after shaking at room temperature for 1 h in 2.5% Triton X-100. The incubation of gel in 0.1 M glycine NaOH, pH 8.3 was done for 3 h at 37° C. Finally, the gel was stained and de-stained by standard coomassie brilliant blue (R-250) dying system. In contrast to blue-paint background the location of the proteolytic zone of activity was not stained by dye.

The one-step purification procedure was performed using HiTrap Benzamidine FF (high sub) column. The column is specified for the purification of serine proteases. The buffers referred to are as follows: 0.05 mMTris HCL, 0.5 M NaCl, PH 7.4 was used as binding and wash buffer. Elution buffers were performed using a step gradient of 0.5 M NaCl, 10 mM HCl, and PH 2.0, and 1 M NaCl, 10 mM HCl, and PH 2.0. Affinity bound substances were eluted by increasing the salt concentration. Step 1: after washing the column with 5 columns of distilled water to remove storage buffer, the column was equilibrated with 5 column volumes of binding buffer. Step 2: cell culture conditioned media was filtered through a 0.45 μm filter and loaded to the column at the same flow rate (1 mL/min). Step 3: the column was again washed with 10 column volumes of binding buffer until no material appeared in eluted fraction. Step 4: the t-PA was eluted from the column with 5 mL of each salt concentration (0.5 and 1 M NaCl) of elution buffers. The pH and conductivity of the buffers were such that the t-PA bound selectively to the column electrostatically while the bulk of the other nonserine protease proteins material did not bind to the resin and were removed in the column flow through. The HiTrap™ Benzamidine FF columns with specification for the purification of serine proteases were utilized for t-PA purification. The buffers referred to be as follows: 0.05 mM Tris-HCL, 0.5 M NaCl, PH 7.4 was used as binding and wash buffer. Elution buffers were performed using a step gradient of 0.5 M NaCl, 10 mM HCl, PH 2.0 and 1 M NaCl, 10 mM HCl, PH 2.0.

Biopool Chromolize t-PA Assay Kit is a biofunctional immunosorbent assay based on capturing t-PA by sp-322 monoclonal antibodies coated on the microtest wells. After fulfilling, the steps from the kit's manual samples were read at 405 nm and 492 nm. Absorbance at 492 nm is measured and subtracted from 405 nm. Various dilutions of each sample were assayed. The amount of developed colour is proportional to the amount of t-PA activity in the sample.

Biopool Chromolize™ t-PA assay Kit with 0.5 unit/ml detection limit was used to measure amidolytic activity quantitatively. The kit is a biofunctional immunosorbent assay based on capturing t-PA by sp-322 monoclonal antibodies coated on the micro test wells. Absorbance at 492 nm is measured and subtracted from 405 nm and the amount of developed color by chromogenic substrate S-2251 is proportional to the amount of t-PA activity in the sample.

The binding of t-PAs was assessed by previously reported methods. Briefly, various concentrations of fibrinogen (0-0.3 mg/mL) were mixed with bovine thrombin (0.5 U/mL) in buffer (0.05 M TrisHCl, pH 7.4, 0.12 M NaCl, 0.01% Tween 80, and 1 mg/mL bovine serum albumin) to form fibrin clots. After incubating the mixture for 30 min at 37° C. equal units (3000 unit) of truncated or full length t-PA were added. The mixture was incubated for 30 min at 37° C., and the clot was removed by centrifugation (15 min, 13 000 rpm, 4° C.; Sigma 202 MD). The amount of enzyme bound to fibrin was calculated from the difference of the total amount of enzyme and the free enzyme in the supernatant, as determined by ELISA. In the ELISA assay, 100 microliters of full length and truncated t-PA are pipetted into each well. The covered plate is incubated for 15 minutes at 37° C., and the reaction is started by the addition of 100 microliters of a plasminogen/S-2251 mixture which contains two parts 10 mM S-2251, one part plasminogen solution (1 mcg/mL), and seven parts assay buffer. The mixture is prepared immediately prior to use. (Assay buffer is Tris-saline prepared by dissolving 605 mg Tris in 800 mL distilled water and adjusting the pH to 8.8 with 6 normal HCl, then dissolving 5.84 grams NaCl and 0.1 grams Triton X-100 into the buffer and diluting to 1000 mL) The absorbance (405 nm) is then measured at 20, 40, and 60 minutes.

Resistance of t-PA to inhibition by PAI-1 was assessed by previously reported methods. Human rPAI-1 in different concentrations from 0 to 128 μM was incubated with full length t-PA or PAI-1 resistant t-PA (in 3,000 IU/ml final concentration) at 25° C. and residual activity was measured at the end of 1 hour incubation period. For the activity measurement the rate of the plasmin generated by the residual full length or truncated-mutant t-PA was measured, in different time intervals. The reaction is started by the addition of 100 μl of plasminogen/S-2251 mixture which is prepared immediately prior to use. The absorbance (405 nm) is then measured at different time intervals. Furthermore, the residual activity was determined using the quantitative ELISA based Chromolize™ t-PA assay kit.

### Results

Double prediction method (DPM) algorithm was used to predict secondary structure of the chimeric truncated t-PA and full length protein. Percentage of alpha helix, extended strand, beta turn and random coil were 27.66, 26.9, 13.45, and 31.9 in chimeric-truncated t-PA and 26.87, 26.69, 15.84, and 30.6 in full-length t-PA, respectively. As could be expected, no significant differences in secondary structure were detected between chimeric truncated t-PA and full-length protein.

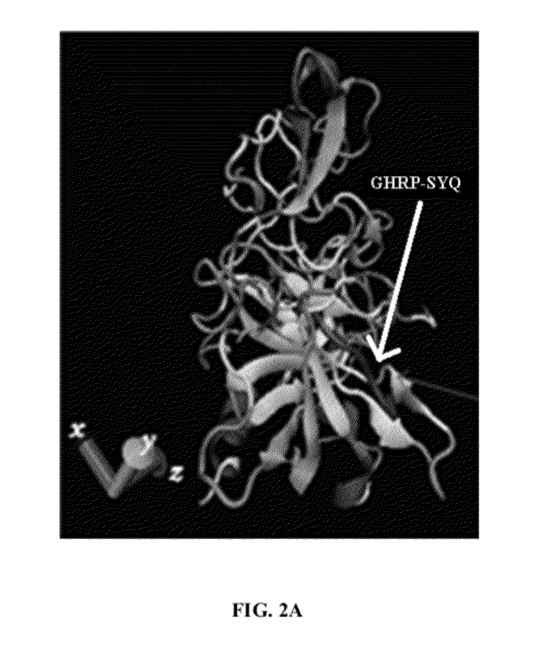

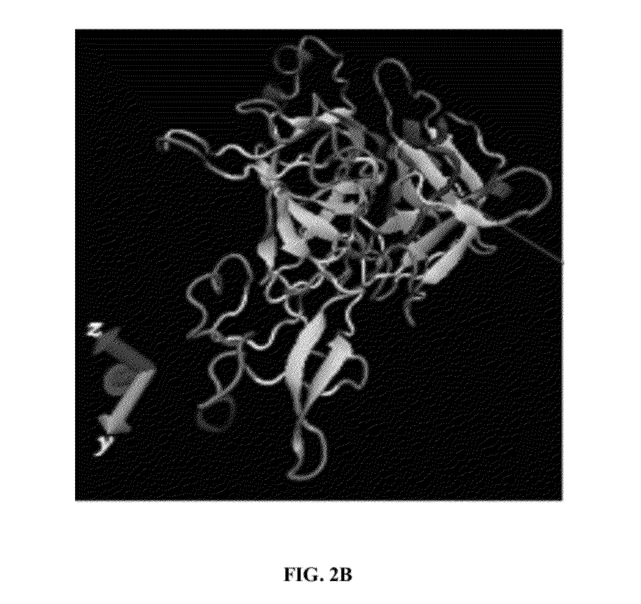

Chimeric-truncated t-PA (GHRP-SYQ-K2S) was successfully modeled via MODELLER 9V1 automodel package using templates. FIG. 2A shows a left back view of a ribbon model of GHRP-SYQ-K2S modeled with MODELLER 9V1 automodel package. In FIG. 2A, the chimeric part is shown. FIG. 2B shows a left bottom view for a ribbon model of GHRP-SYQ-K2S modeled with MODELLER 9V1 automodel package. In FIG. 2B, the purple: alpha helix, blue: 3_{—}10 helix, yellow: extended beta, tan: bridge beta, cyan: turn, and white: coil. FIGS. 2A and 2B are the cartoon ribbon display in VMD 1.8.7 program showing the two different views of GHRP-SYQ-K2S modeled with MODELLER 9V1. The chimeric-truncated model was superimposed on both, K2 (1TPK) and S (1BDA). The predicted model for chimeric-truncated t-PA in the embodiments herein, showed desirable calculated RMSDs after superimposing to the 1tpk (2.79, 2.64, and 2.94 for A, B, and C chains, respectively) and also 0.53 after superimposing to the 1 bda structure. As RMSD values below 2 were acceptable, the results of 1bda superimposing which represents functional domain of t-PA showed that the above-mentioned genetic manipulations do not change the main structure drastically. Therefore, it is predicted that designed novel molecule would not loose thrombolytic activity. The results of Ramachandran plot analysis showed desirable results of almost 1% of residues in outliner region for both K2S and chimeric form which is acceptable in this software's criteria.

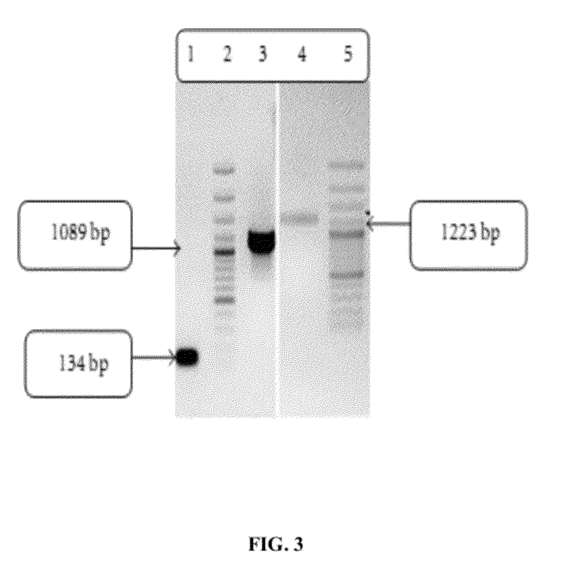

A successful deletion of the first three domains of t-PA (Finger, EGF, and K1) was achieved and also juxtaposing signal peptide upstream of K2S, a 21 base pare DNA sequence, related to GHRP-SYQ amino acids was inserted between signal sequence and K2S domains by using 2 sets of primers in one SOEing reaction, simultaneously. The PCR products for the forward and reverse reactions which were used to generate the chimeric-truncated gene are shown in FIG. 3. FIG. 3 shows a Gel-electrophoresis analysis of PCR-products of Chimeric truncated t-PA. The gel electrophoresis was done on 1% agarose gel. With respect to FIG. 3, Lane 1 shows 134 bp for Forward PCR product i.e. Signal Sequence+GHRP, Lane3 shows 1089 bp for Reverse PCR product i.e. GHRP-SYQ-K2S, and Lane4 shows 1223 bp for Full length SOEn PCR product i.e. signal sequence+GHRP-SYQ-K2S, and Lane 2 & 5 are 1 kb ladder. The PCR products with 134 bp (lane1), 1089 bp (lane3), and 1223 bp (lane4) represent signal sequence−GHRP, GHRP-SYQ-K2S and full-length mutated sequence (signal sequence+GHRP+SYQ+K2S), respectively. These findings are in accordance with the theoretical calculated lengths.

The full-length fused fragment was successfully amplified and ligated into the pTracer-SV40 vector (Invitrogen-USA) using EcoRV and BglII restriction enzyme sites on vector and inserted gene, respectively. Furthermore, ligation was confirmed by digestion with restriction endonucleases. To confirm absence of any kind of mutation during gene manipulation and also to determine desired chimeric conjunction, the cloned gene was sequenced. The sequence showed the desired chimeric conjunction at the 36th to the 39th residue, and first 3 amino acids of native t-PA, exactly before K2S fragment and just after signal sequence.

After transfection of CHO cells with nonlinear plasmid, the cells were allowed to recover for 24 to 48 hours and then assayed by fluorescence microscopy. The results showed 10%-20% transfection efficiency.

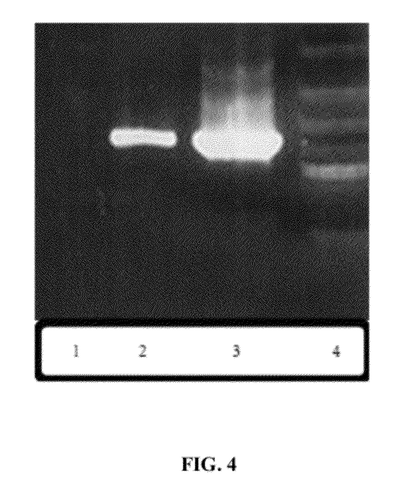

As mentioned, chimeric truncated t-PA was cloned in pTracer-SV40 expression vector for transformation. The positive clones were selected based on Zeocin resistance and PCR amplification of the integrated t-PA gene was performed. The proper band related to chimeric truncated t-PA gene is shown in FIG. 4. FIG. 4 shows a result of Gel-electrophoresis analysis of PCR products performed on stable transfected CHO cells and control cell line. With respect to FIG. 4, Lane 1 shows the PCR product from non-transfected CHO genomic DNA. Lane 2 shows the PCR product from transfected CHO genomic DNA. Lane 3 shows the PCR product from another transfected CHO genomic DNA. Lane 4: 1 kb ladder.

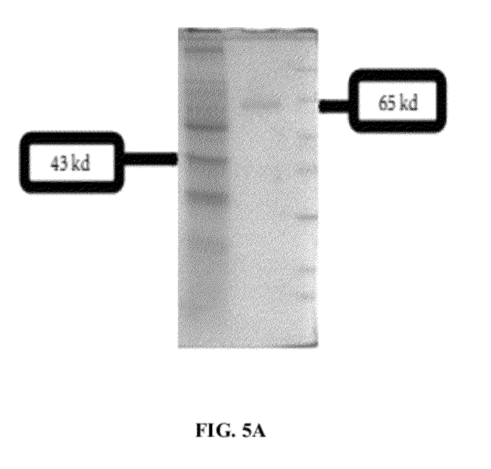

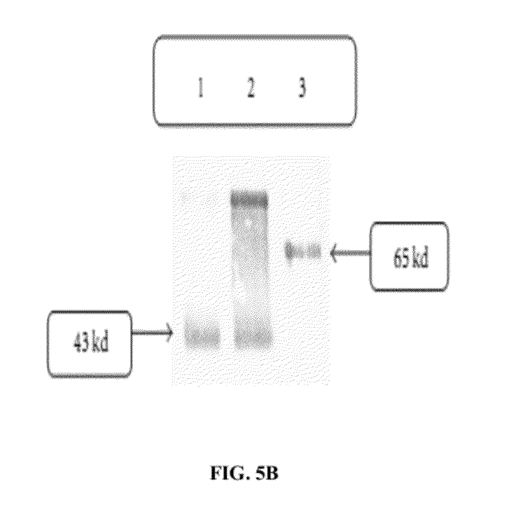

FIG. 5A shows the results of a SDS-PAGE analysis from supernatant of transfected CHO cells, 12% gel-coumasei blue stained. With respect to FIG. 5A, the Right line shows truncated t-PA CHO cell culture medium. Middle line shows full length t-PA and the Left line shows protein marker. Expression of chimeric truncated t-PA gene is shown in FIG. 5A, a 43 kd protein bond is shown in lane 1. This band is related to chimeric truncated t-PA based on theoretical calculations, 359 amino acid sequences. The full-length t-PA protein is shown in lane 2, 65 kDa. This finding was confirmed using antibody against t-PA protein in Western blot analysis, FIG. 5B. FIG. 5B shows Western Blot analysis from supernatant of transfected CHO cells. With respect to FIG. 5B, Lane 1 & 2 shows for chimeric truncated t-PA CHO cell culture medium and Lane 3 for full length t-PA. Lane 1 and 2 in FIG. 5B belong to different clones of the chimeric truncated t-PA, and lane 3 is commercially available t-PA, Actylase.

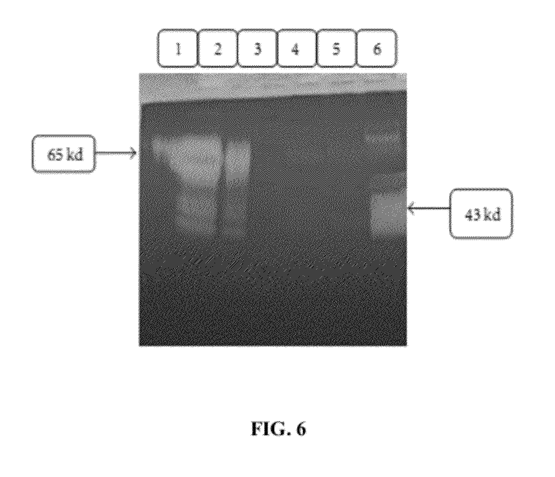

Using the standard electrophoretic procedure, the supernatants of cultured media of transfected CHO cells showed enzymatic activity as clear zones of proteolysis against a blue background of un-degraded stained gelatin. FIG. 6 shows the electrophoretic activity analysis of the transfected t-pa to identify plasminogen activators and proteases in 11% polyacrylamide gel containing gelatin. The gel contains 11% polyacrylamide, 0.1% S DS, 0.1% copolymerized gelatin and 13 μg/ml plasminogen. The electrophoreses of the samples were carried at a constant current of 8 mA and at a temperature of 4° C. With respect to FIG. 6, Lane 3 shows a band for Reference protein mixture, lane 1 for Actylase (full length t-PA), lane 2 for Actylase+polyclonal anti-t-PA rabbit antibody, lane 4 for negative control, lane 6 for transfected CHO cells supernatant, and lane 5 for transfected CHO cells supernatant+anti-t-PA antibody. After electrophoresis, the gels were washed in Triton X-100, incubated in glycine-NaOH buffer at 37° C. for 3 h and fixed, stained, and destained as described under Method. As shown in FIG. 6, clear zone for truncated form is similar to what is demonstrated in the positive control well, commercial t-PA medicine.

The utilized affinity purification procedure resulted in more than 80% purified samples. For quantitative determination of human tissue plasminogen activator activity in purified supernatant samples of transfected CHO cell, a biofunctional immunosorbent assay was performed. Based on Biopool's Chromolize t-PA assay kit, amidolytic unit on days 7 and 9 of culture was found to be 540 unit/mL and 752 unit/mL, respectively. This expression is much higher than what has been shown previously in E. coli, 7 unit/mL. The estimated specific activity was 566,917 unit/mg. This activity is comparable to what is reported for full-length t-PA: 580,000 unit/mg and truncated form: 575,000 unit/mg.

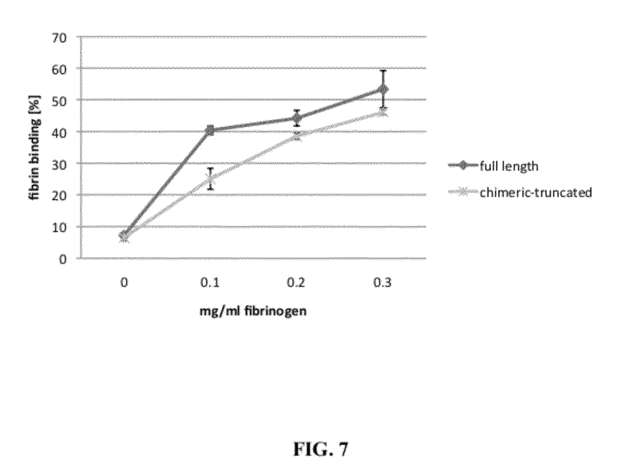

FIG. 7 shows a graphical representation of the fibrin affinity assay of full length and chimeric truncated t-pa. With respect to FIG. 7, different concentrations of fibrin were incubated with full length (⋄) or chimeric-truncated t-PA ( ) Data represent means obtained from three experiments. As it is illustrated in FIG. 7, in 0.2 mg/mL fibrinogen concentration the chimeric truncated protein (GHRP-SYQ-t-PA) showed a fibrin affinity of 38%, compared to full length form with 44%. In 0.3 mg/mL fibrinogen, chimeric-truncated form had 46% affinity to fibrin compared to full-length form with the value of 53%. Therefore, by assuming that the control full-length form has 100% of fibrin affinity, the chimeric-truncated form represented 86% of fibrin affinity of full length protein.

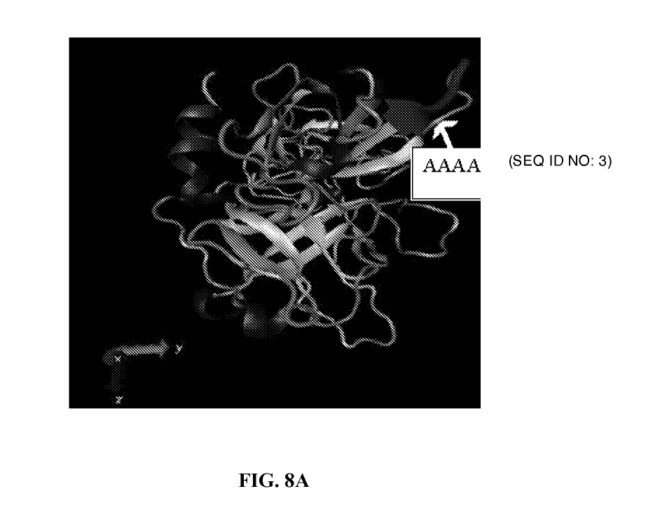

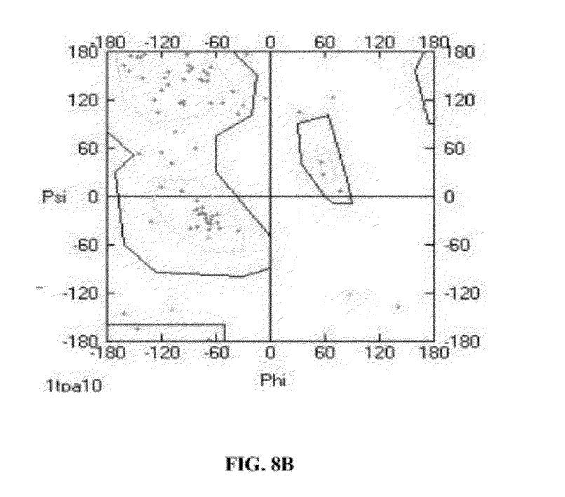

FIG. 8A shows Cartoon Ribbon display in VMD 1.8.7 program for modified t-PA modeled with MODELLER 9V1. With respect to FIG. 8A, left top back view for a ribbon model of modified t-PA modeled with MODELLER 9V1-auto model package can be seen showing the AAAA amino acid residue. Distribution of alpha helix, extended strand, beta turn and random coil percentage in PAI resistant t-PA were 27.41, 26.9, 13.45, 32.23 compared to 26.87, 26.69, 15.84, 30.6 in native full length t-PA. The differences in secondary structure were not significant. The protein model (predicted by Modeller 9V1) was superimposed both on K2 (1TPK) and S (1BDA) PDB templates. The results of superimposing on 1bda, the functional domain of t-PA, showed the RMSD value of 0.3 Å which was in acceptable criteria (Å≦2). FIG. 8B shows the Ramachandran plot analysis for the modified t-PA modeled with MODELLER 9V1. Therefore, the mentioned gene manipulations do not change the main structure drastically. The results of Ramachandran plot analysis also showed 3.1% of residues in outlier region; another confirmation of model validity.

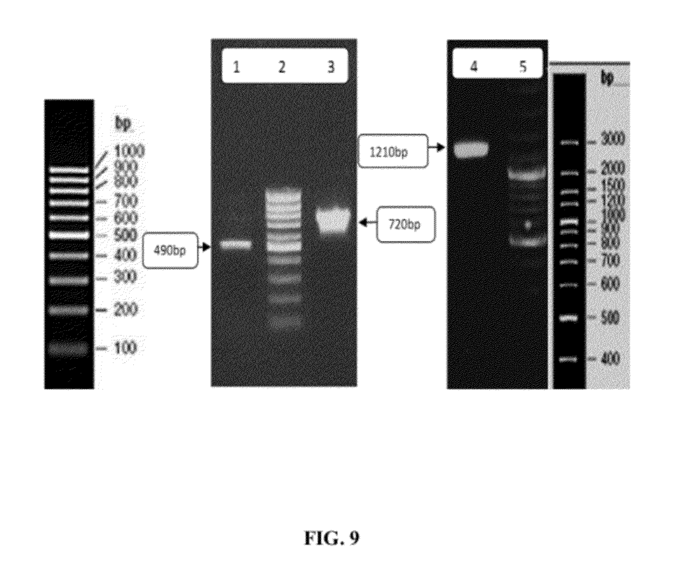

FIG. 9 shows Gel-electrophoresis analysis of PCR-products of Chimeric-Mutant t-PA on 1% agarose gel. With respect to FIG. 9, Lane1 shows 490 bp Upstream PCR product, Signal Sequence, K2 domain and S domain (residues 1-34). Lane 3 shows 720 bp Downstream PCR product, S Domain (residues 32-266). Lane 4 shows 1210 bp Final SOEn PCR product signal sequence, K2 and mutated S domain. Lane 2 & 5 shows a DNA ladder. In accordance with the theoretical calculated lengths, the PCR products with 490 bp, 720 bp and 1,210 bp represent upstream fragment PCR product, downstream fragment PCR product and full length SOEn PCR product respectively. The upstream fragment consists of signal sequence, K2 domain and S domain (up to residue 163). The downstream fragment is composed of, S Domain (residues 161-394); and fused SOEn PCR product includes signal sequence, K2 and mutated S domain. The full length fragment was ligated into the pTracer SV40 vector (Invitrogen-USA) using EcoRV and BglII restriction enzyme sites on the vector and gene of interest, respectively. Further confirmation was done by restriction analysis and sequencing.

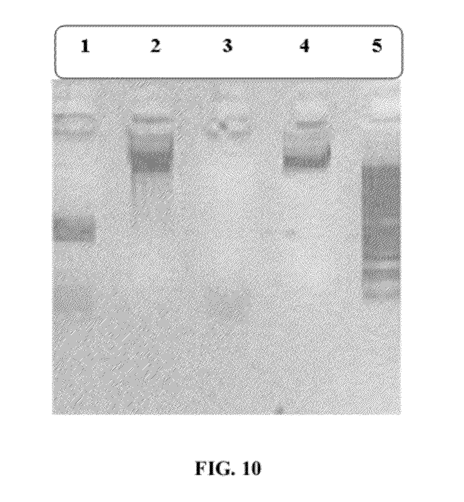

FIG. 10 shows Gel-electrophoresis analysis of PCR-products performed on stable transfected CHO cells and control cell line. With respect to FIG. 10, Lane 1: PCR product from transfected CHO genomic DNA. Lane2: Genomic DNA of transfected CHO cells, Lane3: PCR product from non-transfected CHO genomic DNA. Lane 4: Genomic DNA of non-transfected CHO cells. Lane5: 1 kb ladder. The CHO DG44 cells were transfected with circular plasmid. Afterwards the cells were allowed to recover for 48 hours and then assayed for GFP expression by fluorescence microscopy. The transfection efficiency was about 20%. For stable expression CHO cells were transfected with linearized plasmid. The positive clones were selected under stringent selection pressure of Zeocin™ for several months. PCR amplification with t-PA specific primers was performed on genomic DNA of CHO in order to trace the integrated t-PA gene during 6 months post transfection. The proper 1,210 bp band related to truncated-mutant t-PA gene was a confirmation for the presence of gene in hosts genomic DNA.

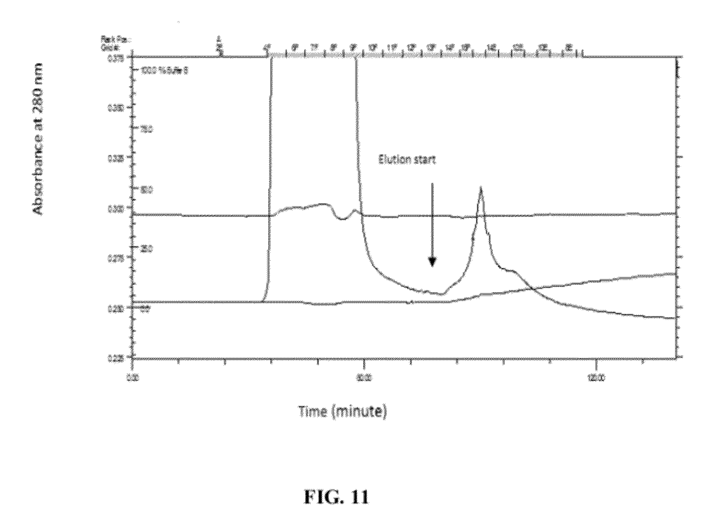

FIG. 11 shows a result of purification of chimeric truncated mutant t-PA on affinity chromatography HiTrap™ Benzamidine FF column. The flow rate was 1 ml/min and 5 ml fractions were collected. Elution start with low pH elution buffer (10 mM HCl, 1 M NaCl, pH 2.0) is marked by (i). The absorbance at 280 nm was recorded and the activity of 5 ml fractions was measured by using the Chromolise™ t-PA assay kit.

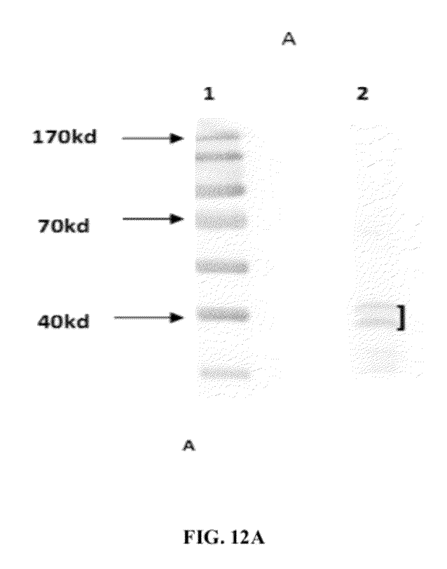

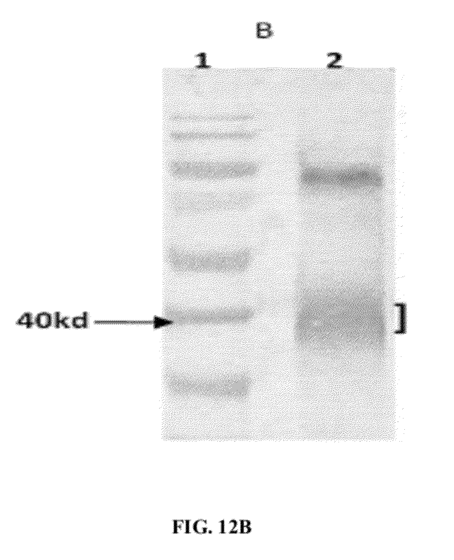

SDS-PAGE and Western Blot analysis using antibody against t-PA, was performed on purified protein. FIG. 12A shows SDS-PAGE analysis from purified truncated-mutant t-PA. FIG. 12A represents bonds related to truncated-mutant t-PA. With respect to FIG. 12A, 12% EBT Silver stained, Lane 2 shows band for Purified truncated-mutant t-PA and Lane 1 is band for Protein marker SM0671. The bands are in accordance with theoretically calculated sizes after signal sequence removal; 359 amino acids (39 KDa) and another glycosylated form with 43 KDa. This was confirmed by Western Blot analysis in FIG. 12B. FIG. 12B shows Western Blot analysis of purified truncated-mutant t-PA. With respect to FIG. 12B, Lane 2 shows band for Truncated-Mutant t-PA and Lane 1 shows band for Protein marker SM0671. These findings were similar to that reported by Burck et al.

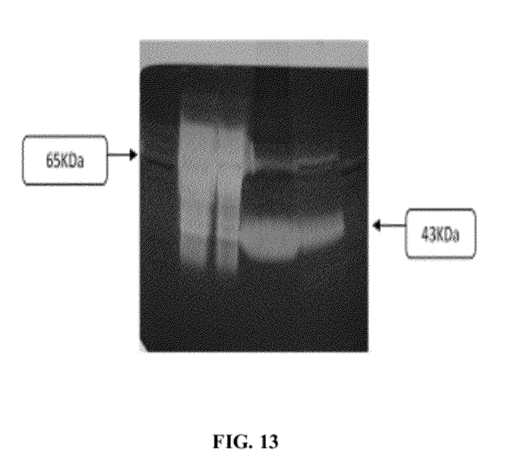

FIG. 13 shows Gelatin hydrolysis assay for plasminogen activators in 11% polyacrylamide gel containing gelatin and plasminogen. With respect to FIG. 13, Lane 2 & 3 show bands for Actylase® (full length t-PA), and Lane 4 & 5 show bands for Transfected CHO cells supernatant, and Lane 1 shows bands for Non transfected CHO cells supernatant, and Lane 6 shows band for protein marker SM0671. After electrophoresis the gels were washed in Triton X-100, incubated in glycine-NaOH buffer at 37° C. for 3 h and fixed, stained, and destained as described under Method. As shown in FIG. 13, clear zones of proteolysis were obvious for both truncated-mutant form and commercial t-PA medicine (Alteplase®). Although, this test is not capable of revealing the exact size of the serine proteases but differences between various sizes of proteins are distinguishable. As seen in FIG. 13, truncated-mutant t-PA and full length t-PA show different places of proteolytic zone on the gel but they are both active.

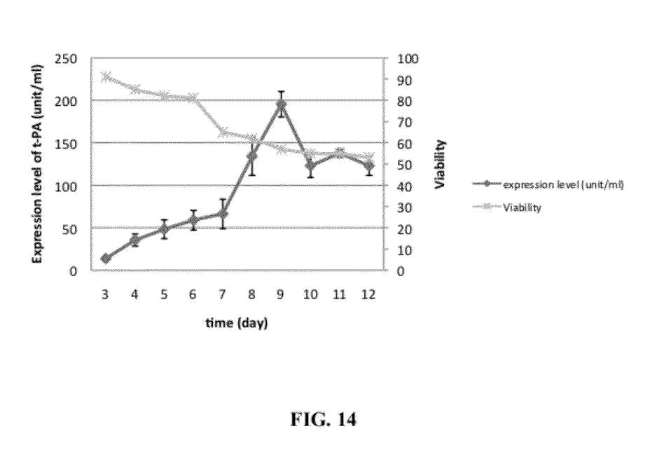

FIG. 14 shows an expression of truncated-mutant t-PA in CHO DG44 supernatant versus viability and Amidolytic assay to determine the expression kinetic of modified t-PA. Expression level data represent means obtained from three experiments. Expression level of CHO cells was determined during days 3 to 12 post culture in batch culture system with 0.3×10⁶ per ml starting cell density. The highest expression level was obtained at day 9, with respect to FIG. 14. It is worth mentioning that the viability reduced from 98% at day first to 65% at day 9. The best expression level was determined to be 750 unit/ml on day 9 of culture in an optimized condition with 0.6 million starting cell density. The estimated specific activity was 570 IU/μg. This activity is comparable to what is reported for full length t-PA: 580 IU/μg and truncated form: 575 IU/μg.

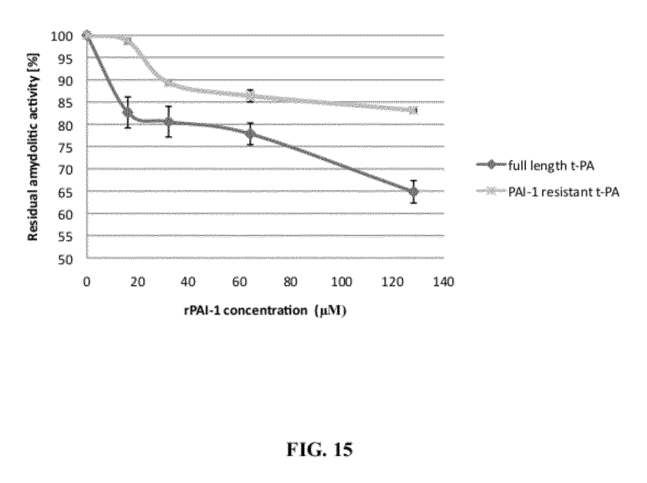

The inhibitory activity of human rPAI-1 toward full length t-PA and truncated-mutant t-PA was determined in vitro. FIG. 15 shows the residual activity of full length and PAI-1 resistant Truncated Mutant t-PA after inhibition by rPAI-1. Full length and truncated forms (3000 IU/ml, final) were incubated for 1 hour at 25° C. with rPAI-1 (16 to 128 μM). As shown in FIG. 15, PAI-1 resistant t-PA represented partial neutralization of activity (85% residual activity in 128 μM PAI) whereas the full length form was more neutralized in similar concentrations (65% of activity was preserved).

Plasminogen activators (PAs) are used in treating cardiovascular and cerebrovascular obstructions. Apart from laborious production and refolding process, short plasma half life has complicated full-length t-PA's clinical application. Deletion-variants of t-PA such as Lanotaplase and Reteplase are nonglycosylated truncated forms constructed to increase plasma half life. It seems that deletion of these regions could be an advantage. In addition, presence of GHRP amino acids sounds beneficial for improving fibrin affinity. Taking advantage of these two factors a chimeric truncated form of t-PA was designed based on in silico analysis. The analysis showed that the molecule preserves the crucial regions of full length t-PA. As previously reported the first 3 amino acids of t-PA (SYQ) are important for the protein activity, and so conjugated this part to the K2S (functional domain) the same as what is done in Reteplase (truncated form of t-PA). This truncated form has proven to be active while SYQ is juxtaposed to K2S domain without any other interfering amino acids. So, it was proposed that SYQ-K2S arrangement is responsible for the serine protease activity. On the other hand, the aim of the embodiments herein, is to improve some pharmacodynamic properties of an existing truncated t-PA (Reteplase). So, all other parts are kept intact; that is, SYQ-K2S are been persevered to observe the sole effect of GHRP addition in fibrin-binding affinity. The chimeric GHRP is designed to increase fibrin affinity compared to Reteplase. In order to have better access to fibrin cloths in purpose of improved interaction of the chimeric protein with fibrin cloths it is better to have the chimeric GHRP situated in the N-terminus and not between other residues the same as what is done in Jiao et al.'s article.

Apart from cell line development and synergism strategies improving efficacy by protein engineering is greatly promising. Principal focus of these efforts is eliminating regions to reduce the size of molecule and increase half life. Deleting F, E, and k1 domains reduces both fibrin binding and fibrin enhancement of plasminogen activator. In order to compensate this disadvantage, inserting additional fibrin-binding sites into the molecule enhances specificity. The Ramachandran plot analysis is represented in Swiss-PDB viewer program and amino acids appear as little cross. The chimeric-truncated form showed desirable protein stability based on Ramachandran plot predictions and RSMD values. Nearly, 1% of the residues, including residues 4 and 5 were found to be in outliner region. Given that the residues 4 and 5 are representing an unnatural, chimeric junction, less than 1% of residues are not modeled well which is favorable compared to similar strategies. Additionally, the best RMSD value was the result of superimposing the model in the embodiments herein, on 1 BDA template which consists of 300 amino acids; 76% of total protein residues and the active site as well.

The experimental analysis confirmed the in silico analysis findings. Chimeric-truncated t-PA was expressed in CHO DG44 cells. The level of expression was found to be 752 IU/mL in optimized condition. This level of expression is promising compared to enzymatic activity of produced t-PA in E. coli (3-7 IU/mL), non-modified rCHO (50 IU/mL), Aspergillus nidulans (0.1 μg/mL), or Leishmania tarentolae (70 IU/mL).

Enhancing productivity of cell lines is crucial for reducing costs. Among nonrecombinant mammalian cell producers, Bowes melanoma cell line is perhaps the best choice with the productivity more than 30 IU per 106 cells per day (0.1 mg/L conditioned medium). Epithelial cells like guinea pig ear keratocytes (GPK) have yields of production ranged over 5-10 mg/L from perfusion cultures with (7-10)×10⁶ anchored cells per milliliter. Between recombinant mammalian cells with inserted copies of human t-PA gene, mouse C127 fibroblasts (with 55 mg/L production rate), rat myeloma (with 52 mg/L production rate), and CHO cells (with 65 mg/L production rate) in perfused or fed-batch systems are worth mentioning.

Considering other recombinant cultures, Saccharomyces cervisiae has a low yield of production with extensive hyper glycosylated and heterogenic forms. Bacterium Escherchia coli produces t-PA as inclusion bodies consisting 5%-10% of cell proteins. Because of low recovery of active t-PA from inclusion bodies, bacterial production process does not seem feasible. In one study, co-expressing of native or heterologous cysteine oxidoreductases increased expression levels to 180 μg/L. Despite the complexity of renaturation from inclusion bodies, E. coli fermentation is far less demanding than animal cell culture. Furthermore, glycosylation is not necessary for biological activity; also non-glycosylated forms show increased plasma half lives, hence, t-PA.

Production in bacteria is still desirable and perhaps become feasible either with advances in renaturation methods or with reduction in number of disulfide bonds via protein engineering. The suggested chimeric-truncated form is promising due to potential of production in prokaryotic systems similar to its non-glycosylated commercial relative, Reteplase.

According to the embodiments herein, a cassette gene was designed by having t-PA signal sequence upstream of chimeric-truncated molecule via SOEing PCR in order to take advantage of proper folding and glycosylation in CHO. A Different size of templates is a problematic matter in applying SOEing PCR procedure and closer template sizes are preferred in this strategy. Here, owing to appropriate primer design, two remarkably different sizes of templates, 134 bp and 1089 bp, were successfully sued by using sensible molar ratios of both templates and primers. In addition, deletion and insertion were performed concurrently.

The protein migrated with an apparent molecular mass of 43 kDa on SDS-PAGE; also, an 86 kDa bond was obvious while western blotting was performed. All the dimer form was converted to monomer form by dissolving the protein in 8 M urea, and just one bond at 43 KD was observed on Western Blot. These data confirmed that the doubled molecular mass size could be due to aggregation of truncated t-PA.

Biological activity was tested with electrophoretic zymography analysis. Previous studies have shown that the main extracellular protease band secreted by the CHO was approximately 92 or 95 KDa. This indicates that the extra higher size bonds are probably from CHO itself. However, presence of an obvious band of almost 43 KDa and its drastic diminish after inhibition by antibody confirms that the proteolytic activity was exclusively due to t-PA and not other probably existing serine proteases. As it is shown previously, (lane 5 of FIG. 6), significant decline of proteolytic zone in the presence of special t-PA antibody is apparent. The degree of inhibition by antibody is dependent upon the t-PA concentration, but is nevertheless dramatically reduced when compared to the equivalent concentration in samples without antibody.

Both fibrin specificity and fibrin affinity are touted as desirable properties in PAs. The fibrin-specific agents are much more likely to bind to fibrin bound plasminogen as opposed to free plasminogen. Therefore, the lack of systemic plasminemia limits systemic fibrinogenolysis, which is associated with bleeding complications. Reteplase was initially thought to have desired fibrin-specificity, but in practice it was less fibrin specific than full-length form. Subsequently, higher frequency of bleeding was reported by Reteplase. Previous studies showed that Reteplase has the in vitro fibrin binding equal to 30% of that of full-length form. The fibrin-binding experiment demonstrated that chimeric-truncated t-PA had 86% of fibrin-binding capacity of full-length form, Actylase, suggesting that the novel protein in the embodiments herein could be targeted to fibrin clot with GHRP.

Aiming at further improvement in pharmacodynamic properties of the protein, a PAI-1 resistant novel form of truncated t-PA was designed based on In Silico modeling and then successfully expressed in CHO DG44. Targeting mutations made in this project are intended to increase the resistance of truncated t-PA to inhibition by PAI-1.

Plasminogen activators play an important role in treating cardiovascular and cerebrovascular obstructions. Short plasma half life due to the need for Infusion administration, has complicated full length t-PA's clinical application. Improving efficacy by protein engineering is greatly promising. Principal focus of these efforts is eliminating first domains of native t-PA which are involved in hepatic clearance to reduce the size of molecule and increase half life. Deletion variants of t-PA such as Reteplase® are not resistant to inhibition by PAI-1. On the other side, mutation in amino acids involved in the interaction with PAI-1 from KHRR to AAAA sounds beneficial for generating resistance to PAI-1. Taking advantage of these two factors a truncated-mutant form of t-PA that is resistant to PAI-1 inhibition activity was designed based on In Silico analysis.

In Silico analysis showed that the molecule preserves the secondary and 3D structure of active site regions of wild type t-PA. In Ramachandran plot analysis, nearly, 3% of the residues were found to be in outlier region which is favorable compared to similar strategies. Furthermore, the best RMSD value (0.3 Å) was the result of superimposing our model on 1 bda template which includes 76% of total protein residues and the active site as well.

Truncated-mutant t-PA was expressed in CHO DG44 cells. The level of expression was found to be 750 IU/ml based on quantitative amidolytic activity assay. This level of expression is promising compared to enzymatic activity of produced t-PA in E. coli (3-7 IU/ml), non-modified rCHO (50 IU/ml), Aspergillus nidulans (0.1 μg/ml) (27) or Leishmania tarentolae (70 IU/ml). The specific activity of truncated-mutant t-PA (570 IU/μg) is comparable to full length t-PA (580 IU/μg) and truncated form (575 IU/μg).

As glycosylation is not necessary for biological activity, t-PA production in bacteria is still desirable and perhaps become feasible with reduction in number of disulfide bonds via protein engineering. The suggested PAI-1 resistant truncated-mutant form is promising due to potential of production in prokaryotic systems similar to its non-glycosylated commercial relative, Reteplase®.

The protein migrated with molecular weights of 39 and 43 KDa on SDS-PAGE which was expected due to glycosylation heterogeneity in CHO expression system. An 86 kDa bond was obvious while Western Blotting was performed. The conversion of dimer form to monomer form by dissolving the protein in 8 M urea was a confirmatory data to the fact that the doubled molecular mass size was due to partial aggregation of truncated-mutant t-PA.

Biological activity was tested with electrophoretic zymography analysis. The main extracellular protease secreted by the CHO is approximately 92 or 95 KDa. Therefore, the presence of an obvious band of almost 43 KDa confirms that the proteolytic activity was exclusively due to truncated-mutant t-PA and not other probably existing serine proteases.

A PAI-1 resistant variant of t-PA is preferred because of in-creased patency as a fibrinolytic agent especially toward plate-let rich thrombi, since high levels of PAI-1 contribute to the re-occlusion. The amino acids involved in PAI-1 and t-PA interaction are residues 296 to 304 from t-PA and three acidic residues (Glu350, Glu351 and Asp355) from PAI-1. Substitution or deletion in these critical regions has been reported to enhance resistance to PAI-1 inhibition considerably.

In the present study, a truncated-mutant variant of t-PA was constructed by mutation of residues 128-131 from KHRR to AAAA performed on truncated form of t-PA from our previous work. The biochemical, biological and thrombolytic properties of this protein was confirmed with different qualitative and quantitative analysis. Purified mutant t-PA mut (K128-R131) was obtained with an amidolytic activity of 570 IU/μg against a chromogenic substrate for t-PA which is comparable to the full-length t-PA; Alteplase (Actilyse)® with 580 IU/μg specific activity.

In agreement with previous findings, the inhibition rate of mutant t-PA mut (K128-R131), by PAI-1 in purified systems was lower than that of full length t-PA. Reduction of the amidolytic activity to 65% was achieved by the addition of the same concentration of rPAI-1 (128 μM) for full length t-PA, compared to 85% for mutant t-PA confirming the partial resistance of the mutant to PAI-1. These results suggest that, in the presence of PAI-1, the thrombolytic potency of mutant t-PA mut (K128-R131) is superior to that of full length t-PA, as a result of resistance to PAI-1. Whether the clinical use of this PAI-1-resistant t-PA mutant would lead to less PAI-1-mediated re-occlusion after thrombolytic or with a better thrombolytic potency towards clots still has to be investigated.

Deletion variants of t-PA have the advantage of fewer disulfide bonds in addition to higher plasma half lives. A new variant was developed by deletion of the first three domains in t-PA in addition to substitution of KHRR 128-131 amino acids with AAAA in truncated t-PA. The specific activity of this new variant, 570 IU/μg, was found to be similar to those found in full length t-PA (Alteplase), 580 IU/μg. A 65% and 85% residual activity after inhibition by rPAI-1 was observed for full length and truncated-mutant form, respectively. This new variant as the first PAI-1 resistant truncated t-PA may offer more advantages in clinical conditions in which high PAI-1 levels makes the thrombolytic system prone to re-occlusion.

Apart from deleting the first three domains of t-PA and adding a chimeric tetra-peptide Gly-His-Arg-Pro (GHRP) with high fibrin affinity upstream of K2S to compensate for reduction of fibrin affinity due to finger domain deletion, aiming at further improvement in pharmacodynamic properties of the protein, a PAI-1 resistant novel form of truncated t-PA was designed and then successfully expressed in CHO DG44. Targeting mutations made in the final variant are intended to increase the resistance of truncated t-PA to inhibition by PAI-1.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A chimeric truncated and mutant variant of a tissue plasminogen activator (t-pa) comprising:
a native t-pa deleted with a first three domains, wherein the first three domains are Finger domain (F), a Growth Factor domain (EGF) and a Kringle 1 domain (K1);
a chimeric tetrapeptide, wherein the chimeric tetrapeptide is Gly-His-Arg-Pro (GHRP), wherein the chimeric tetrapeptide is at a position of 36 to 39 amino acid of the mutant variant; and
a substituted amino acids at a position of 128-131, wherein the substituted amino acids are AAAA amino acids;
wherein the mutant variant is resistant to plasminogen activator inhibitor-1.

2. The chimeric truncated and mutant variant of t-pa according to claim 1, wherein the mutant variant has 394 amino acids.

3. The chimeric truncated and mutant variant of t-pa according to claim 1, wherein the chimeric tetrapeptide (GHRP) is situated on the N-terminus.

4. The chimeric truncated and mutant variant of t-pa according to claim 1, wherein the mutant variant has a fibrin affinity of 86%.

5. The chimeric truncated and mutant variant of t-pa according to claim 1, wherein the mutant variant has a specific activity of is 570 IU/μg.

6. The chimeric truncated mutant variant of t-pa according to claim 1, wherein the mutant variant has a residual activity of 85% after inhibition by plasminogen activator inhibitor-1.

7. A method for preparing a chimeric truncated and mutant variant of t-pa comprises:
deleting a first three domains of a native t-pa, wherein the first three domains of the native t-pa are a Finger domain (F), a Growth Factor domain (EGF) and a Kringle 1 domain (K1);
adding a chimeric tetrapeptide, wherein the chimeric tetrapeptide is Gly-His-Arg-Pro (GHRP), wherein the chimeric tetrapeptide is added at a position of 36 to 39 amino acid of the mutant variant; and
substituting amino acids KHRR with amino acids AAAA, wherein the substitution is made at position of 128-131 amino acid of the native t-pa.

8. The method according to claim 7, wherein the mutant variant is produced by using a Splicing by Overlap Extension PCR (SOEing-PCR) method.

9. The method according to claim 7, wherein the chimeric tetrapeptide (GHRP) is added upstream to a kringle 2 domain and a serine protease domain (i.e. K2S domain) of the native t-pa.

10. The method according to claim 7, wherein the chimeric tetrapeptide compensates for a diminished fibrin affinity due to F domain deletion from a native t-pa.

11. The method according to claim 7, wherein the mutant variant has 394 amino acids.

12. The method according to claim 7, wherein the mutant variant has a fibrin affinity of 86%.

13. The method according to claim 7, wherein the mutant variant has a specific activity of 570 IU/μg.

14. The method according to claim 7, wherein the mutant variant has a residual activity of 85% after inhibition by plasminogen activator inhibitor-1.

15. A chimeric truncated and mutant variant of a tissue plasminogen activator (t-pa) comprising:
a native tissue plasminogen activator (t-pa) having a signal sequence domain, followed by a chimeric tetrapeptide, followed by a tripeptide, followed by a kringle 2 domain, followed by a serine protease domain; and
a substituted amino acids at position 128-131, wherein the substituted amino acids are AAAA amino acids, and wherein the mutant variant is resistant to plasminogen activator inhibitor-1.

16. The chimeric truncated and mutant variant of a tissue plasminogen activator (t-pa) according to claim 15, wherein the chimeric tetrapeptide is Gly-His-Arg-Pro (GHRP) at a position of 36 to 39 amino acid of the mutant variant.

17. The chimeric truncated and mutant variant of a tissue plasminogen activator (t-pa) according to claim 15, wherein the tripeptide is Ser-Tyr-Glu (SYQ).

18. The chimeric truncated and mutant variant of t-pa according to claim 15, wherein an arrangement of the tripeptide, the kringle 2 domain and the serine protease domain (SYQ-K2S) is responsible for the serine protease activity.
